(19) United States
(12) Patent Application Publication
(10) Pub. No.: US 2019/0038637 A1
(43) Pub. Date: Feb. 7, 2019

(54) Use of Hydroxyprogesterone Derivatives for Enhanced Health and Physical Performance II

(71) Applicant: Eric Marchewitz, Shelby Township, MI (US)

(72) Inventor: Eric Marchewitz, Shelby Township, MI (US)

(21) Appl. No.: 15/669,909

(22) Filed: Aug. 5, 2017

(51) Int. Cl. A61K 31/57; A61K 9/00

(52) U.S. Cl. CPC A61K 31/57; A61K 9/0014; A61K 9/0053; A61K 9/006

(57) ABSTRACT

A method for the use of derivatives of hydroxyprogesterone (17-alpha-hydroxypregn-4-ene-3,20-dione) to enhance health and physical performance in humans and more particularly to the use of hydroxyprogesterone derivatives for restoring renal hormonal balance, decreasing body weight, reducing adipose tissue, increasing endurance, promoting skeletal muscle growth, boosting androgen levels, inhibiting aromatase, and increasing cognitive function.

### REFERENCE TO RELATED APPLICATIONS

This application is a divisional of U.S. patent application Ser. No. 13/506,148 filed Mar. 30, 2012. This application claims the benefit of the filing date of U.S. Provisional Application No. 61/517,934, filed Apr. 28, 2011, and the entirety of which is incorporated herein by reference.

### FIELD OF THE INVENTION

This invention discloses a method for the use of derivatives of hydroxyprogesterone (17-alpha-hydroxypregn-4-ene-3,20-dione) to enhance health and physical performance in humans and more particularly to the use of hydroxyprogesterone derivatives for restoring renal hormonal balance, decreasing body weight, reducing adipose tissue, increasing endurance, promoting skeletal muscle growth, boosting androgen levels, inhibiting aromatase, and increasing cognitive function.

### BACKGROUND OF THE INVENTION

The adrenal gland produces many steroid hormones. These steroid hormones play a major role in many bodily processes including, for example, maintaining healthy renal hormonal balances, promoting skeletal muscle growth, facilitating red blood production (erythropoiesis), regulation of glucose and insulin levels and controlling cellular aging. The steroids produced by the adrenal gland can generally be divided into three groups. These include glucocorticoids, which influence carbohydrate metabolism, mineralocorticoids, which control electrolyte and water balance, and sex steroid hormones.

The first group, Glucocorticoids, such as for example, cortisol, regulate the breakdown, or catabolism, of skeletal muscle proteins into amino acids. These amino acids are then transported to the liver and converted into glucose during gluconeogenesis. Excessive amounts of glucocorticoids can result in higher blood glucose and insulin levels and can contribute to increased body fat and type II diabetes. Glucocorticoids are also known to play a role in the aging process by increasing cellular and mitochondrial breakdown in the body as well as inducing tissue inflammation. Examples of glucocorticoids include prednisone, prednisolone, methylprednisolone, dexamethasone, hydrocortisone (cortisol), and cortisone.

The second groups of adrenal steroids, mineralocorticoids, help the body to retain sodium and water. However, excessive amounts of mineralocorticoids can result in hypertension and cardiovascular disease. Examples of mineralocorticoids include aldosterone and fludrocortisone.

The third groups of adrenal steroids commonly referred to as sex hormones include DHEA, androgens, estrogens, progesterone, and hydroxyprogesterone. Adrenal androgens oppose the actions of glucocorticoids and result in skeletal muscle anabolism, reductions in blood glucose and insulin levels, and a reduction in body fat. Hydroxyprogesterones have shown active and pro-hormonal properties, meaning that physiological effects are induced as a result of progesterone, as well as metabolites of the compound.

With normal younger adults, all three groups of adrenal steroids are produced in a healthy balance. However, as people age, many of these steroids decline in production, for example, DHEA and progesterone, damaging the optimum hormonal equilibrium as a result of higher concentrations of other adrenal steroids such as, for example, cortisol, in relation to the declining steroids.

Utilizing dietary supplements to restore balance are known to have beneficial effects. Hydroxyprogesterone derivatives are more beneficial than oral progesterone because oral progesterone can be metabolized into glucocorticoids as well as estrogens via enzymatic activity. Hydroxyprogesterone have increased resistance to the enzymes in these metabolic pathways—due to changes in functional group binding—and are therefore less likely to result in negative side effects. Furthermore, hydroxyprogesterone derivatives act directly on the androgen receptor. For example, the 5-alpha-reduced derivative of 17-hydroxyprogesterone is resistant to conversion to estrogen or estradiol, therefore preventing any of the negative side effects associated with increased estrogen. This is a result of the change in the shape of the steroidal structure which inhibits the enzymatic activity of aromatase. In addition, because of the resistance to metabolizing and converting into estrogenic compounds, the 5-alpha-reduced derivative is more likely to be converted into a more positively active compound via the enzymatic activity of CYP 17a1 and 17-alpha-hydroxysteroid dehydrogenase, such as androgens. There exist a need for safer and more beneficial adrenal steroid supplements such as hydroxyprogesterone derivatives to promote health and hormonal equilibrium in humans.

### SUMMARY OF THE INVENTION

In accordance with a first aspect, a method is disclosed for administering a hydroxyprogesterone derivative or a physiologically acceptable salt, ester, ether, or pegylation thereof for the purpose of optimizing hormonal balance, decreasing body weight, reducing adipose tissue, increasing endurance, and promoting skeletal muscle growth

From the foregoing disclosure and the detailed description set forth below of various preferred embodiments of the invention it will be apparent to those skilled in the art that the disclosed invention provides a significant advance in the methods of administering compounds for promoting health in humans. Additional features and advantages of various preferred embodiments will be better understood in view of the detailed description provided below.

### DETAILED DESCRIPTION

It will be apparent to those skilled in the art, that is, to those who have a knowledge or experience in this area of technology that many variations are possible for the method of administering hydroxyprogesterone derivatives for enhancing health and physical performance. The following detailed discussion of various alternative and preferred features and embodiments of the disclosed invention will illustrate the general principles of the invention with reference to improved methods of enhancing physical health and performance by administering hydroxyprogesterone derivatives as a dietary supplement. Other embodiments suitable for other applications will be apparent to those skilled in the art given the benefit of this disclosure.

As used herein, a derivative of a compound refers to a species having a chemical structure that is similar to the compound, yet containing a chemical group not present in the compound and/or deficient of a chemical group that is present in the compound. The substance to which the derivative is compared is known as the “parent” substance. Here, for example, the parent compound is the adrenal steroid hydroxyprogesterone. A derivative may be made by modification of the parent compound or by synthesis from other starting materials that are not similar to the parent.

In accordance with conventional steroid carbon numbering, an atom or functional group attached to a ring depicted herein is termed α (denoted by a dashed triangular shaped bond) if it lies below the plane of the paper or β (denoted by a solid triangular shaped bond) if it lies above the plane of the paper. When R1, or R2, have one functional group listed, it is understood that the fourth bond to the carbon is hydrogen, and that carbon is in an opposing position to the noted bond. Other related methods of use for hydroxyprogesterone derivatives suitable for optimizing hormonal balance, decreasing body weight, reducing adipose tissue, increasing endurance, promoting skeletal muscle growth inhibiting aromatase, and increasing cognitive function will be apparent to those with a thorough understanding of biochemical mechanisms, given the benefit of this disclosure.

The hydroxyprogesterone derivatives disclosed herein can be active both after metabolism as well as without metabolism. Additionally, the disclosed derivatives can inhibit the aromatase that can occur with adrenal steroids that converts these steroids to estradiol and estrogen, thereby optimizing hormone balance levels and promoting other beneficial effects including decreasing body weight, reducing adipose tissue, increasing endurance, and promoting skeletal muscle growth.

The disclosed invention includes groupings of derivatives of hydroxyprogesterone within several general formulas. In one embodiment the hydroxyprogesterone derivatives might be derived from the general grouping of hydroxypregna-1-enes within the following formula:

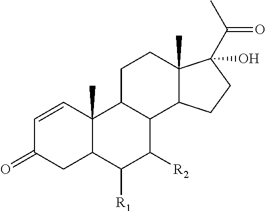

Where R1 is of H, α-OH, β-OH, or O; R2 is one of H, α-OH, β-OH, or O. Hydroxyprogesterone derivatives made according to the above formula can consist of 17-alpha-hydroxypregn-1-ene-3,20-dione; 7-alpha,17-alpha-dihydroxypregn-1-ene-3,20-dione; 7-beta,17-alpha-dihydroxypregn-1-ene-3,20-dione; 17-alpha-hydroxypregn-1-ene-3,7,20-trione; 6-alpha,17-alpha-dihydroxypregn-1-ene-3,20-dione; 6-alpha,7-alpha,17-alpha-trihydroxypregn-1-ene-3,20-dione; 6-alpha,7-beta,17-alpha-trihydroxypregn-1-ene-3,20-dione; 6-alpha,17-alpha-dihydroxypregn-1-ene-3,7,20-trione; 6-beta,17-alpha-dihydroxypregn-1-ene-3,20-dione; 6-beta,7-alpha,17-alpha-trihydroxypregn-1-ene-3,20-dione; 6-beta,7-beta,17-alpha-trihydroxypregn-1-ene-3,20-dione; 6-beta,17-alpha-dihydroxypregn-1-ene-3,7,20-trione; 17-alpha-hydroxypregn-1-ene-3,6,20-trione; 7-alpha,17-alpha-dihydroxypregn-1-ene-3,6,20-trione; 7-beta,17-alpha-dihydroxypregn-1-ene-3,6,20-trione; 17-alpha-hydroxypregn-1-ene-3,6,7,20-tetraone.

In an alternative embodiment the hydroxyprogesterone derivatives might be derived from the general grouping of the hydroxy-5-alpha-pregnanes within the following formula:

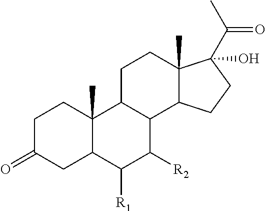

Where R1 is of H, α-OH, β-OH, or O; R2 is one of H, α-OH, β-OH, or O. Hydroxyprogesterone derivatives made according to the above formula can consist of 17-alpha-hydroxy-5-alpha pregna-3,20-dione; 7-alpha,17-alpha-dihydroxy-5-alpha-pregna-3,20-dione; 7-beta,17-alpha-dihydroxy-5-alpha-pregna-3,20-dione; 17-alpha-hydroxy-5-alpha-pregna-3,7,20-trione; 6-alpha,17-alpha-dihydroxy-5-alpha-pregna-3,20-dione; 6-alpha,7-alpha,17-alpha-trihydroxy-5-alpha-pregna-3,20-dione; 6-alpha,7-beta,17-alpha-trihydroxy-5-alpha-pregna-3,20-dione; 6-alpha,17-alpha-dihydroxy-5-alpha-pregna-3,7,20-trione; 6-beta,17-alpha-dihydroxy-5-alpha-pregna-3,20-dione; 6-beta,7-alpha,17-alpha-trihydroxy-5-alpha-pregna-3,20-dione; 6-beta,7-beta,17-alpha-trihydroxy-5-alpha-pregna-3,20-dione; 6-beta,17-alpha-dihydroxy-5-alpha-pregna-3,7,20-trione; 17-alpha-hydroxy-5-alpha-pregna-3,6,20-trione; 7-alpha,17-alpha-dihydroxy-5-alpha-pregna-3,6,20-trione; 7-beta,17-alpha-dihydroxy-5-alpha-pregna-3,6,20-trione; 17-alpha-hydroxy-5-alpha-pregna-3,6,7,20-tetraone.

In another alternative embodiment the hydroxyprogesterone derivatives might be derived from the general grouping of hydroxypregna-1,4-dienes within the following formula:

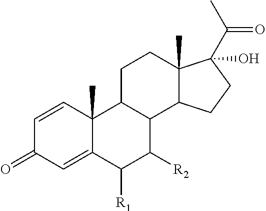

Where R1 is of H, α-OH, β-OH, or O; R2 is one of H, α-OH, α-OH, or O. Hydroxyprogesterone derivatives made according to the above formula can consist of 17-alpha-hydroxypregna-1,4-diene-3,20-dione; 7-alpha,17-alpha-dihydroxypregna-1,4-diene-3,20-dione; 7-beta,17-alpha-dihydroxypregna-1,4-diene-3,20-dione; 17-alpha-hydroxypregna-1,4-diene-3,7,20-trione; 6-alpha,17-alpha-dihydroxypregna-1,4-diene-3,20-dione; 6-alpha,7-alpha,17-alpha-trihydroxypregna-1,4-diene-3,20-dione; 6-alpha,7-beta,17-alpha-trihydroxypregna-1,4-diene-3,20-dione; 6-alpha,17-alpha-dihydroxypregna-1,4-diene-3,7,20-trione; 6-beta,17-alpha-dihydroxypregna-1,4-diene-3,20-dione; 6-beta,7-alpha,17-alpha-trihydroxypregna-1,4-diene-3,20-dione; 6-beta,7-beta,17-alpha-trihydroxypregna-1,4-diene-3,20-dione; 6-beta,17-alpha-dihydroxypregna-1,4-diene-3,7,20-trione; 17-alpha-hydroxypregna-1,4-diene-3,6,20-trione; 7-alpha,17-alpha-dihydroxypregna-1,4-diene-3,6,20-trione; 7-beta,17-alpha-dihydroxypregna-1,4-diene-3,6,20-trione; 17-alpha-hydroxypregna-1,4-diene-3,6,7,20-tetraone.

In another alternative embodiment the hydroxyprogesterone derivatives might be derived from the general grouping of hydroxypregn-4-ene-3,20-dione within the following formula:

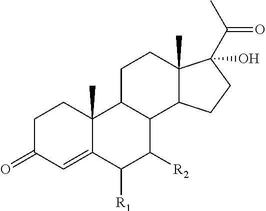

Where R1 is of H, α-OH, β-OH, or O; R2 is one of H, α-OH, β-OH, or O. Hydroxyprogesterone derivatives made according to the above formula can consist of 17-alpha-hydroxypregn-4-ene-3,20-dione; 7-alpha,17-alpha-dihydroxypregn-4-ene-3,20-dione; 7-beta,17-alpha-dihydroxypregn-4-ene-3,20-dione; 17-alpha-hydroxypregn-4-ene-3,7,20-trione; 6-alpha,17-alpha-dihydroxypregn-4-ene-3,20-dione; 6-alpha,7-alpha,17-alpha-trihydroxypregn-4-ene-3,20-dione; 6-alpha,7-beta,17-alpha-trihydroxypregn-4-ene-3,20-dione; 6-alpha,17-alpha-dihydroxypregn-4-ene-3,7,20-trione; 6-beta,17-alpha-dihydroxypregn-4-ene-3,20-dione; 6-beta,7-alpha,17-alpha-trihydroxypregn-4-ene-3,20-dione; 6-beta,7-beta,17-alpha-trihydroxypregn-4-ene-3,20-dione; 6-beta,17-alpha-dihydroxypregn-4-ene-3,7,20-trione; 17-alpha-hydroxypregn-4-ene-3,6,20-trione; 7-alpha,17-alpha-dihydroxypregn-4-ene-3,6,20-trione; 7-beta,17-alpha-dihydroxypregn-4-ene-3,6,20-trione; 17-alpha-hydroxypregn-4-ene-3,6,7,20-tetraone.

In another alternative embodiment the hydroxyprogesterone derivatives might be derived from the general grouping of hydroxypregna-1,4,6-triene within the following formula:

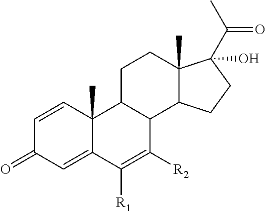

Where R1 is of H, α-OH, β-OH, or O; R2 is one of H, α-OH, β-OH, or O. Hydroxyprogesterone derivatives made according to the above formula can consist of 17-alpha-hydroxypregna-1,4,6-triene-3,20-dione; 7-alpha,17-alpha-dihydroxypregna-1,4,6-triene-3,20-dione; 7-beta,17-alpha-dihydroxypregna-1,4,6-triene-3,20-dione; 17-alpha-hydroxypregna-1,4,6-triene-3,7,20-trione; 6-alpha,17-alpha-dihydroxypregna-1,4,6-triene-3,20-dione; 6-alpha,7-alpha,17-alpha-trihydroxypregna-1,4,6-triene-3,20-dione; 6-alpha,7-beta,17-alpha-trihydroxypregna-1,4,6-triene-3,20-dione; 6-alpha,17-alpha-dihydroxypregna-1,4,6-triene-3,7,20-trione; 6-beta,17-alpha-dihydroxypregna-1,4,6-triene-3,20-dione; 6-beta,7-alpha,17-alpha-trihydroxypregna-1,4,6-triene-3,20-dione; 6-beta,7-beta,17-alpha-trihydroxypregna-1,4,6-triene-3,20-dione; 6-beta,17-alpha-dihydroxypregna-1,4,6-triene-3,7,20-trione; 17-alpha-hydroxypregna-1,4,6-triene-3,6,20-trione; 7-alpha,17-alpha-dihydroxypregna-1,4,6-triene-3,6,20-trione; 7-beta,17-alpha-dihydroxypregna-1,4,6-triene-3,6,20-trione; 17-alpha-hydroxypregna-1,4,6-triene-3,6,7,20-tetraone.

In a preferred embodiment the compound derived from the hydroxypregna-1-enes grouping set forth above of hydroxyprogesterone derivatives, would be 17-alpha-hydroxypregn-1-ene-3,20-dione of the general formula:

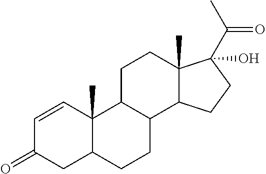

In an alternative preferred embodiment the compound derived from the hydroxy-5-alpha-pregnanes grouping set forth above of hydroxyprogesterone derivatives, would be 17-alpha-hydroxy-5-alpha-pregna-3,20-dione of the general formula

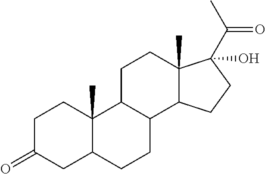

In another alternative preferred embodiment the compound derived from the hydroxypregna-1,4-dienes grouping set forth above of hydroxyprogesterone derivatives, would be 17-alpha-hydroxypregna-1,4-diene-3,20-dione of the general formula:

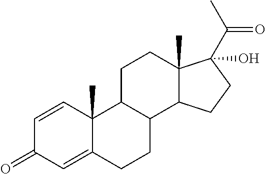

In another alternative preferred embodiment the compound derived from the hydroxypregna-1,4,6-triene grouping set forth above of hydroxyprogesterone derivatives, would be 17-alpha-hydroxypregna-1,4,6-triene-3,20-dione

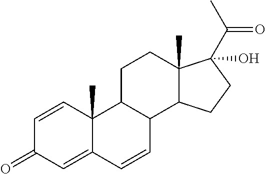 [SMILES: CC(=O)[C@@]1(O)CCC2C3C=CC4=CC(=O)C=C[C@]4(C)C3CC[C@@]21C]

In another alternative preferred embodiment the compound derived from the hydroxypregn-4-ene-3,20-dione grouping set forth above of hydroxyprogesterone derivatives, would be 17-alpha-hydroxypregn-4-ene-3,20-dione

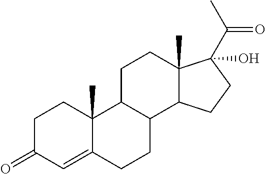 [SMILES: CC(=O)[C@@]1(O)CCC2C3CCC4=CC(=O)CC[C@]4(C)C3CC[C@@]21C]

All of the hydroxyprogesterone derivative compounds disclosed herein could be administered either orally, topically, transdermally, intranasally, by injection, sublingually, and/or transrectally. In the preferred embodiment they might be administered orally. In one alternative they might be administered sublingually. In one alternative embodiment they might be delivered via a liquid sublingual method. In another alternative embodiment they might be administered through a topical application. In the preferred embodiment they would preferably be administered orally mixed with solid or liquid carriers in appropriate unit doses.

The preferred amount of the active ingredient that is to be administered would depend on various factors such as the age and weight of the user. An effective oral daily dosage of the described hydroxyprogesterone derivatives might comprise a daily dose of 5 to 1000 mg. In the preferred embodiment the dose might be 40-300 mg daily. In an alternative embodiment the doses might be delivered in two daily doses. In another embodiment it might be four doses. Any amount of dosage could be utilized however that would achieve the objective of promoting health and physical performance however. In one alternative the preferred method of delivery might be to administer the oral dose as a soft gelatin capsule or oral liquid suspension, either in one or more doses per day. The hydroxyprogesterone derivatives as disclosed herein might also be mixed with other dietary supplements, binders, and/or excipients.

### Example 1

A capsules containing 17-alpha-hydroxy-5-alpha-pregna-3,20-dione, or another similar hydroxyprogesterone derivative, is mixed with microcrystalline cellulose or other non-active carriers and placed into a hard-gelatin capsule. Each capsule contains 100 mg of 17-alpha-hydroxy-5-alpha-pregna-3,20-dione and is directed to be taken 3-6 times per day.

### Example 2

Tablets containing 17-alpha-hydroxy-5-alpha-pregna-3,20-dione or another similar hydroxyprogesterone derivative is mixed with microcrystalline cellulose or other non-active carrier and pressed into a hard tablet. Each tablet contains 100 mg of 17-alpha-hydroxy-5-alpha-pregna-3,20-dione and is directed to be taken 3-6 times per day.

### Example 3

A liquid concentration containing 17-alpha-hydroxy-5-alpha-pregna-3,20-dione or another similar hydroxyprogesterone derivative is mixed with hydropropyl-beta-cyclodextrin and water. Each dose contains 20 mg of 17-alpha-hydroxy-5-alpha-pregna-3,20-dione and directed to be taken 2-6 times per day.

### Example 4

A soft gel containing 17-alpha-hydroxy-5-alpha-pregna-3,20-dione or another similar hydroxyprogesterone derivative is mixed with oil-based or other carriers and placed into a soft gelatin capsule. Each capsule contains 100 mg of 17-alpha-hydroxy-5-alpha-pregna-3,20-dione and is directed to be taken 2-6 times per day.

### Example 5

A topical solution containing 17-alpha-hydroxy-5-alpha-pregna-3,20-dione or another similar hydroxyprogesterone derivative is mixed with a solvent, carrier, or other liquid delivery mechanism. Each dose contains 20-100 mg of 17-alpha-hydroxy-5-alpha-pregna-3,20-dione and is directed to be applied topically 1-4 times per day.

From the foregoing disclosure and detailed description of certain preferred embodiments, it will be apparent that various modifications, additions, and other alternative embodiments are possible without departing from the true scope and spirit of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A method of administering to a human a hydroxyprogesterone derivative or a physiologically acceptable salt, ether, or pegylation thereof as a compound that provides at least one of anti-aging renal hormonal balance, decreased body weight, reduction of adipose tissue, increased endurance, and skeletal muscle growth, of the general formula:

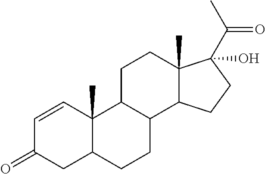

2. The method of claim 1 wherein the hydroxyprogesterone derivative is administered in one of the following ways: orally, topically, transdermally, intranasally, by injection, sublingually, and transrectally.

3. The method of claim 1 wherein the hydroxyprogesterone derivative is administered as a daily dosage between about 5 mg and 600 mg.

4. The method of claim 1 wherein the hydroxyprogesterone derivative is administered as a daily dosage between about 40 mg and 300 mg.

5. The method of claim 1 wherein the hydroxyprogesterone derivative is administered as a daily dosage between about 20 mg and 150 mg twice a day.

6. The method of claim 1 wherein the hydroxyprogesterone derivative is administered as a daily dosage between about 10 mg and 75 mg four times per day.

7. The method of claim 2 wherein the hydroxyprogesterone derivative is administered in the form of a liquid sublingual.
